(19) United States
(12) Patent Application Publication
(10) Pub. No.: US 2026/0137337 A1
(43) Pub. Date: May 21, 2026

(54) CHARACTERIZATION OF SLEEP MOTION ACTIVITY

(71) Applicant: Hoffmann-La Roche Inc., Little Falls, NJ (US)

(72) Inventors: Leo Stefan GSCHWIND, Basel (CH); Vitaliy KOLODYAZHNIY, Riehen (CH)

(73) Assignee: Hoffmann-La Roche Inc., Little Falls, NJ (US)

(21) Appl. No.: 19/417,997

(22) Filed: Dec. 12, 2025

Related U.S. Application Data
(63) Continuation of application No. PCT/EP2024/066716, filed on Jun. 17, 2024.

(30) Foreign Application Priority Data
Jun. 19, 2023 (EP) 23179987.5

(51) Int. Cl. A61B 5/00; A61B 5/11

(52) U.S. Cl. CPC A61B 5/4815; A61B 5/1102; A61B 5/1118; A61B 5/1126; A61B 5/4082; A61B 5/7246; A61B 5/7253; A61B 5/7271

(57) ABSTRACT

The present invention relates to systems and methods for analyzing data from motion activity monitoring technology. It is particularly, but not exclusively, concerned with a method for determining motion activity patterns during sleep.

### FIELD OF INVENTION

The present invention relates to systems and methods for analyzing data from motion activity monitoring technology. It is particularly, but not exclusively, concerned with a method for determining motion activity patterns during sleep.

### BACKGROUND TO THE INVENTION

Sleep disturbances are among the most common symptoms occurring in patients with neurological dysfunctions. A poor sleep quality is also often associated with the patient performing regular or irregular movements during the sleep.

Several technologies exist that can be used to diagnose sleep disorders. Polysomnography, for example, is a sleep study that records physiological changes that occur during sleep, in terms of brain activity, eye movements, heart rhythm and muscle activity. The downside of polysomnography is that it is invasive and is typically performed for a few nights and only in a dedicated environment. On the contrary, actigraphy is an activity monitoring technique using a non-invasive device, usually a wrist-worn wearable sensor, which allows continuous measurement of motor activity in a clinical or non-clinical setting. Actigraphy has been used to assess sleep/wake behaviours in subjects suffering from insomnia, Alzheimer, dementia, asthma, COPD (chronic obstructive pulmonary disease), headache disorders. Existing methods used to analyze actigraphy data (Long et al, 2017; Granovsky et al, 2018) are able to perform a sleep/wake classification based on motion activity. However, such methods fail to characterize sleep motion activity patterns and to correlate sleep motion activity patterns with a medical condition.

Therefore there is a need for improved systems and methods for analyzing data from motion activity monitoring technology, and in particular, but not exclusively, for determining motion activity patterns during sleep.

### STATEMENTS OF INVENTION

The present invention relates to systems and methods for analyzing data from motion activity monitoring technology. It is particularly, but not exclusively, concerned with a method for determining motion activity patterns during sleep. The methods can find particular use in the analysis of sleep motion activity patterns of subjects with a neurological dysfunction, such as for example Angelman Syndrome (AS). The methods can be used, among other applications, to diagnose a neurological dysfunction, to monitor and/or predict disease progression, to assess and/or predict the response of patients to a treatment, to compare the sleep motion activity patterns of a subject at a plurality of time points. These methods can also be used to compare the sleep motion activity patterns of different subjects diagnosed with the same or similar medical conditions, to compare the sleep motion activity patterns of a subject diagnosed with a medical condition and those of a healthy subject, to identify cohorts of patients with similarities in their sleep motion activity patterns, to identify and/or predict subpopulations that might benefit more from a given treatment.

The present inventors have identified that prior art methods for analyzing data from motion activity monitoring technology are insufficient to determine sleep motion activity patterns because said methods are confined to the classification of sleep and wake phases based on the motion activity. Moreover, prior art methods suffer from a bias towards motion signals with high intensity that tend to dominate the motion activity at the expense of low-intensity signals and small variations thereof.

Furthermore, prior art methods rely on absolute intensity values which do not allow comparisons neither of motion activities of a subject at different time points nor of motion activities across subjects.

The invention according to the present application discloses the surprising effect that by leveraging specific operations on the signals received from the one or more motion activity monitoring systems, such as for example aggregations, transformations and normalizations, it is possible to determine sleep motion activity patterns and/or their likelihood of occurrence. The invention according to the present application further discloses that by applying a logarithmic transform to the signals it is possible to reduce the bias of high-intensity motion signals and therefore to reveal otherwise hidden sleep motion activity patterns in the low-activity intensity range.

Thus, according to a first aspect, the present invention provides a computer-implemented method of processing signals from one or more motion activity monitoring systems to determine sleep motion activity patterns of a subject, the method comprising the steps of: receiving signals from the one or more motion activity monitoring systems within a predefined time window, wherein the one or more motion activity monitoring systems comprise actigraph units and/or ballistocardiogram sensors; dividing the predefined time window in epochs; computing, from the received signals, activity levels in one or more epochs; converting the computed activity levels to decibels (dB); selecting a bin width in dB; obtaining an activity level profile as a histogram of the dB-converted activity levels binned with the selected bin width; optionally normalizing the obtained activity level profile; determining, based on the optionally normalized activity level profile, the sleep motion activity patterns of the subject. The subject can be a human subject. The subject can be an adult subject. The subject can be a paediatric subject. The subject can be a healthy subject. The subject can be a subject that has been diagnosed having a disease or disorder or being likely to have a disease or disorder. In particular, the subject can be a subject that has been diagnosed with autism disorder, Angelman Syndrome (AS), Alzheimer's disease, Parkinson's disease, Multiple Sclerosis (MS), Amyotrophic Lateral Sclerosis (SLA), Spinal Muscular Atrophy (SMA), Huntington's disease. In particular, the subject can be a subject likely to have autism disorder, Angelman Syndrome (AS), Alzheimer's disease, Parkinson's disease, Multiple Sclerosis (MS), Amyotrophic Lateral Sclerosis (SLA), Spinal Muscular Atrophy (SMA), Huntington's disease. The epochs can comprise time slots of duration between 1 second and 60 seconds, in particular 4 seconds. Depending on the predefined time window, the number of epochs defines the time resolution. The higher the number of epochs, the higher the time resolution. The predefined time window can comprise a full bed time window (i.e. “night”) of the subject, or a fraction thereof. The step of converting the computed activity levels to dB can comprise converting activity level x using the transform 10*log₁₀ (αx+β), in particular wherein α=β=1. This avoids the singularity of the logarithmic function at x=0. The typical range of activity levels, thus of dB, depend on the specific motion activity monitoring system. A maximum measurable device-specific activity level of 32767 would correspond to circa 45 dB. The step of selecting a bin width in dB can comprise selecting a bin width of 1 dB, of 2 dB, of 5 dB. A meaningful bin width value would depend on the specific motion activity monitoring system. The optional step of normalizing the activity level profile can comprise computing the normalized activity level profile according to the equation

$${h}^{′} = {( {h}_{1} , … , {h}_{n} )}^{T} / {\sum }_{i = 1}^{n} {h}_{i} ,$$

where hᵢ is the value of the activity level in the i-th bin. Normalizing the activity level profiles allows for comparisons of activity level profiles obtained within different time windows, and/or activity level profiles of different subjects. Normalized activity level profiles contain information about the likelihood of occurrence of motion as a function of the motion activity level.

The step of determining, based on the activity level profile, the sleep motion activity patterns of the subject can comprise measuring the intensity and/or the number of body movements. The step of determining, based on the normalized activity level profile, the sleep motion activity patterns of the subject can comprise measuring the relative intensity and/or the number of body movements. Measuring the relative intensity of body movements can comprise measuring the likelihood of occurrence of said body movements.

The method can have one or more of the following features.

The step of receiving signals from the one or more motion activity monitoring systems within a predefined time window, wherein the one or more motion activity monitoring systems comprise actigraph units and/or ballistocardiogram sensors, can comprise resampling the received signals. As an example, the received signals can be resampled to a sampling frequency between 30 and 200 Hz, in particular but not exclusively 50 Hz for actigraph units or 100 Hz for ballistocardiogram sensors. The step of receiving signals from the one or more motion activity monitoring systems within a predefined time window can further comprise aggregating the received signals. For example, when the received signals are 3D signals, the received signals can be aggregated in 1D by means of a projection along one dimension, or by means of selecting the dimension that contains more information, or by other aggregation means, in particular by calculating a Euclidean norm, i.e. \sqrt{x²+y²+z²}, wherein x, y, z are the orthogonal components of the 3D signals.

The step of computing, from the received signals, activity levels in one or more epochs can comprise computing summarized metrics of the received signals within the one or more epochs. Summarized metrics can comprise mean, median, trimmed versions thereof.

According to a second aspect, the present invention provides a method according to the first aspect, further comprising selecting a plurality of predefined time windows and, for at least two of the selected time windows, performing the steps a. to h. The plurality of predefined time windows can comprise several full bed time windows (i.e. “nights”) of the subject, or fractions thereof. The plurality of predefined time windows can comprise several consecutive full bed time windows of the subjects, or fractions thereof. The plurality of predefined time windows can comprise several non-consecutive full bed time windows of the subjects, or fractions thereof. The selected time windows can comprise, among the plurality of predefined time windows, bed time windows wherein the subjects slept alone. The selected time windows can comprise, among the plurality of predefined time windows, bed time windows wherein the subjects slept with a caregiver. The selected time windows can comprise, among the plurality of predefined time windows, bed time windows of a period in which the subject was undergoing a medical treatment. The selected time windows can comprise, among the plurality of predefined time windows, bed time windows of a period in which the subject was transitioning from a medical treatment to another medical treatment. The selected time windows can comprise, among the plurality of predefined time windows, bed time windows of a period in which the subject was transitioning from no medical treatment to a medical treatment. Performing the steps a. to h. for at least two of the selected time windows can comprise performing the steps a. to h. for two selected time windows. Performing the steps a. to h. for at least two of the selected time windows can comprise performing the steps a. to h. for two consecutive selected time windows. Performing the steps a. to h. for at least two of the selected time windows can comprise performing the steps a. to h. for a year of bed time windows. Performing the steps a. to h. for at least two of the selected time windows can comprise performing the steps a. to h. for six months of bed time windows wherein the subject was not undergoing any medical treatment plus six months of bed time windows wherein the subject was undergoing a medical treatment.

The method can further comprise the steps of: estimating the similarity among the (optionally normalized) activity level profiles and/or among activity levels in predetermined bins of the (optionally normalized) activity level profiles obtained for the at least two selected time windows; and/or identifying variations in the sleep motion activity patterns of the subject determined for the at least two selected time windows. The step of estimating the similarity among the activity level profiles and/or among activity levels in predetermined bins of the activity level profiles can comprise performing one or more similarity measurements comprising cosine similarity measurement, Intersection over Unit measurement, statistical tests, in particular p-value tests, or a combination thereof. Statistical tests can be non-parametric statistical tests, in particular but not exclusively Mann-Whitney (MW) tests and/or Brunner-Munzel (BM) tests. As an example, with a=(a₁, . . . , aₙ)^{T} and b=(b₁, . . . , bₙ)^{T} being two n-dimensional vectors corresponding to two activity level profiles with n bins each, the cosine similarity between the two level activity profiles a and b is performed by computing the dot product of the two vectors divided by the product of their magnitudes: cos(φ)=a·b/(∥a∥∥b∥). As activity level profiles contain only non-negative values, the value of the cosine similarity measurement is between 0 for complete dissimilarity and 1 for complete similarity (i.e. equal profiles). Cosine similarity is also suitable for dimensionality reduction and visualization. The angle φ=arccos[a·b/(∥a∥∥b∥)] between vectors a and b can be used for a 2D visualization of similarity/dissimilarity on a unit circle. The cosine similarity between the two activity profiles a and b in a subset of the n bins of the activity level profiles can be performed by computing the cosine similarity between the two vectors

$${a}^{′} = {( {a}_{1}^{′} , … , {a}_{m}^{′} )}^{T}$$

$$and$$

$${b}^{′} = {( {b}_{1}^{′} , … , {b}_{m}^{′} )}^{T}$$

where m<n bins. As another example, to quantify the difference in specific activity intensity ranges for two groups of p and q individuals, e.g. p patients and q healthy individuals, 160 separate non-parametric statistical tests comparing the two groups for each of the respective activity intensity bins in the respective individual's activity profile can be performed. In particular, the pairs of samples

$${a}_{i} = {( {a}_{i}^{( 1 )} , … , {a}_{i}^{( p )} )}^{T} and {b}_{i} = {( {b}_{i}^{( 1 )} , … , {b}_{i}^{( q )} )}^{T}$$

comprising respectively the values of activity intensity for each of the p patients and q healthy individuals in the activity intensity bin i can be compared with a statistical test. To compare all bins in a specific intensity range where i=1, . . . , n, a plurality of n statistical tests can be performed. The step of estimating the similarity among the activity level profiles and/or among activity levels in predetermined bins of the activity level profiles obtained for the at least two selected time windows can comprise aggregating the activity level profiles obtained for a number of selected time windows and estimating the similarity among the aggregated activity level profiles and/or among activity levels in predetermined bins of the aggregated activity level profiles. For example, the activity level profiles obtained for several nights over a week can be aggregated in a weekly-means distribution. For example, the activity level profiles obtained for several nights over a month can be aggregated in a monthly-means distributions. Similarity can be estimated among weekly-means distributions or monthly-means distributions. The step of identifying variations in the sleep motion activity patterns of the subject can comprise measuring the variation of intensity and/or of the number 175 and/or of the time occurrence of body movements.

The invention according to the present application also discloses the surprising effect that by characterizing sleep motion activity patterns of a subject it is possible to infer information about the subject's general health state, and in particular to diagnose a neurological dysfunction associated with sleep motion activity or to monitor a neurological dysfunction associated with sleep motion activity in a subject already diagnosed as having said dysfunction. For example, sleep motion activity patterns comprising movements in a particular intensity range can reveal a specific medical condition or predict the occurrence of a specific medical condition when compared to reference sleep motion activity patterns. The invention according to the present application also discloses the surprising effect that by characterizing variations of sleep motion activity patterns of a subject it is possible to infer information about the subject's general health state, and in particular to diagnose a neurological dysfunction associated with sleep motion activity or to monitor a neurological dysfunction associated with sleep motion activity in a subject already diagnosed as having said dysfunction. For example, variations of sleep motion activity patterns comprising variations of (relative) intensity and/or of number and/or of time occurrence of movements can reveal a specific medical condition or predict the occurrence of a specific medical condition when compared to reference variations of sleep motion activity patterns.

Thus, according to a third aspect, the present invention provides a method of diagnosing or monitoring a neurological dysfunction associated with sleep motion activity in a subject, the method comprising determining the sleep motion activity patterns of a subject using the method of any embodiment of the preceding aspects, optionally wherein the neurological dysfunction is Angelman Syndrome (AS). Diagnosing a neurological dysfunction associated with sleep motion activity in a subject can comprise determining the sleep motion activity patterns of the subject and comparing them with reference sleep motion activity patterns. Reference sleep motion activity patterns can comprise sleep motion activity patterns of the subject at a different point in time. Reference sleep motion activity patterns can comprise sleep motion activity patterns associated with a healthy population. Reference sleep motion activity patterns can comprise sleep motion activity patterns associated with a healthy subpopulation, for example a subpopulation with a given age range, sex, life style. Monitoring a neurological dysfunction associated with sleep motion activity in a subject can comprise determining the sleep motion activity patterns of the subject and comparing them with reference sleep motion activity patterns. Reference sleep motion activity patterns can comprise sleep motion activity patterns of the subject at a different point in time. Reference sleep motion activity patterns can comprise sleep motion activity patterns associated with a healthy population. Reference sleep motion activity patterns can comprise sleep motion activity patterns associated with a healthy subpopulation, for example a subpopulation with a given age range, sex, life style. Diagnosing a neurological dysfunction associated with sleep motion activity in a subject can comprise determining variations of sleep motion activity patterns of the subject and comparing them with reference variations of sleep motion activity patterns. Reference variations of sleep motion activity patterns can comprise variations of sleep motion activity patterns associated with a healthy population. Reference variations of sleep motion activity patterns can comprise variations of sleep motion activity patterns associated with a healthy subpopulation, for example a subpopulation with a given age range, sex, life style. Monitoring a neurological dysfunction associated with sleep motion activity in a subject can comprise determining variations sleep motion activity patterns of the subject and comparing them with reference variations of sleep motion activity patterns. Reference variations of sleep motion activity patterns can comprise variations of sleep motion activity patterns associated with a healthy population. Reference variations of sleep motion activity patterns can comprise variations of sleep motion activity patterns associated with a healthy subpopulation, for example a subpopulation with a given age range, sex, life style.

According to a fourth aspect, the present invention provides a method of determining whether a subject with a neurological dysfunction associated with sleep motion activity is likely to benefit from a therapy, the method comprising determining the sleep motion activity patterns of a subject using the method of any embodiment of the preceding aspects, optionally wherein the neurological dysfunction is Angelman Syndrome (AS). The therapy can comprise the administration of a sleep drug. The therapy can comprise the administration of a drug for the treatment of the neurological dysfunction or symptoms thereof. For example, a worsening of AS can be associated with an increased sleep motion activity. AS patients for which sleep motion activity patterns show more activity in the high-intensity activity levels or a higher number of body movements are likely to benefit from a drug aimed at decreasing activity levels or at keeping activity levels stable or at decreasing the number of body movements or at keeping the number of body movements stable. As another example, a worsening of Spinal Muscular Atrophy (SMA) or of other neurodegenerative diseases can be associated with a decreased sleep motion activity. SMA patients for which sleep motion activity patterns show a lower activity in the high-intensity activity levels or a lower number of body movements are likely to benefit from a drug aimed at increasing activity levels or at keeping activity levels stable or at increasing the number of body movements or at keeping the number of body movements stable. According to a further aspect, the present invention provides a method of treating a subject, the method comprising: determining whether a subject with a neurological dysfunction associated with sleep motion activity is likely to benefit from a therapy using the method of any embodiment of the fourth aspect; and administering a therapeutically effective amount of the therapy.

The invention according to the present application discloses the surprising effect that by quantifying similarities in sleep motion activity patterns of subjects it is possible to identify cohorts of patients, to identify and/or predict subpopulations that might benefit more from a given treatment. Quantifying similarities in sleep motion activity patterns can comprise estimating the similarity among activity level profiles across subjects. As an example, a specific range of intensities of movements can be indicative of a specific age range of the subjects. As another example, a specific range of intensities of movements can be indicative of the presence of a medical condition or of a medical condition in a specific status. Quantifying similarities in sleep motion activity patterns can comprise estimating the similarity among activity levels in predetermined bins of the activity level profiles. As an example, a specific frequency of movements in a specific range of intensities can be indicative of the presence of a medical condition or of a medical condition in a specific status. Identifying variations in the sleep motion activity patterns also allows to identify cohorts of patients with a similar evolution of a specific medical condition and/or predict which subpopulations that might benefit from a given treatment.

Thus, according to a fifth aspect, the present invention provides a method of identifying one or more subpopulations of subjects with a neurological dysfunction associated with sleep motion activity, the method comprising determining the sleep motion activity patterns of at least two of the subjects using the method of any embodiment of the preceding aspects, and assigning each of the at least two subjects to the one or more subpopulations based on the determined sleep motion activity patterns, optionally wherein the neurological dysfunction is Angelman Syndrome 265 (AS). Assigning each of the at least two subjects to the one or more subpopulations based on the determined sleep motion activity patterns can comprise: estimating the similarity among activity level profiles and/or among activity levels in predetermined bins of the activity level profiles obtained for the at least two subjects; and/or identifying variations in the sleep motion activity patterns determined for the at least two subjects. The step of estimating the similarity among activity level profiles and/or among activity levels in predetermined bins of the activity level profiles can comprise estimating the similarity among activity level profiles and/or among activity levels in predetermined bins of the activity level profiles using the method of any embodiment of the preceding aspects. The step of identifying variations in the sleep motion activity patterns determined for the at least two subjects can comprise identifying variations in the sleep motion activity patterns determined for the at least two subjects using the method of any embodiment of the preceding aspects. As an example, one subpopulation can be identified by assigning to said subpopulation subjects for which the sleep motion activity patterns show enhanced motion activity levels in a given intensity range. Said subpopulation can be for example a group of subjects of the same age range. Said subpopulation can be for example a group of subjects diagnosed with the same neurological dysfunction. Said subpopulation can be for example a group of subjects of the same age range and diagnosed with the same neurological dysfunction.

According to a further aspect, there is provided a system of a processor, and a computer readable medium comprising instructions that, when executed by the processor, cause the processor to perform the computer-implemented steps of the method of any preceding aspect. The system can further comprise one or more motion activity monitoring systems, for example one or more actigraph units and/or one or more ballistocardiograph sensors. According to a further aspect, there is provided a non-transitory computer readable medium or media comprising instructions that, when executed by at least one processor, cause the at least one processor to perform the method of any embodiment of any aspect described herein. According to a further aspect, there is provided a computer program comprising code which, when the code is executed on a computer, causes the computer to perform the method of any embodiment of any aspect described herein.

### BRIEF DESCRIPTION OF THE FIGURES

FIG. 1 illustrates an embodiment of a system that can be used to implement one or more aspects described herein.

FIG. 2 is a flow diagram showing, in schematic form, a method of processing signals from one or more motion activity monitoring systems to determine sleep motion activity patterns of a subject, according to the invention.

FIG. 3 is a flow diagram showing, in schematic form, a method of processing signals from one or more motion activity monitoring systems to determine sleep motion activity patterns of a subject for at least two selected time windows, according to the invention.

FIG. 4 is a flow diagram showing, in schematic form, a method of diagnosing or monitoring Angelman Syndrome in a subject, according to the invention.

FIG. 5 is a flow diagram showing, in schematic form, a method of determining whether a subject with Angelman Syndrome is likely to benefit from a therapy, according to the invention.

FIG. 6 is a flow diagram showing, in schematic form, a method of identifying one or more subpopulations of subjects with Angelman Syndrome, according to the invention.

FIG. 7 shows an example of stable sleep motion activity patterns in an 18-years old subject diagnosed with Angelman Syndrome, according to the invention.

FIG. 8 shows an example of unstable sleep motion activity patterns in a 2-years old subject diagnosed with Angelman Syndrome, according to the invention.

FIG. 9 shows an example of a method of identifying subpopulations of subjects with Angelman Syndrome by determining sleep motion activity patterns of the subjects, according to the invention.

### DETAILED DESCRIPTION

In describing the present invention, the following terms will be employed, and are intended to be defined as indicated below.

A neurological dysfunction associated with sleep motion activity as used herein refers to a neurological condition or disorder that affects brain functions, in particular sleep functions and motor functions, including neurodevelopmental disorders, psychiatric disorders, chronic disorders, neurodegenerative disorders. Examples of neurodevelopmental disorders include autism disorder, Angelman Syndrome (AS). Examples of neurodegenerative disorders include Alzheimer's disease, Parkinson's disease, Multiple Sclerosis (MS), Amyotrophic Lateral Sclerosis (SLA), Spinal Muscular Atrophy (SMA), Huntington's disease.

The systems and method described herein can be implemented in a computer system, in addition to the structural components and user interactions described. As used herein, the term “computer system” includes the hardware, software and data storage devices for embodying a system and carrying out a method according to the described embodiments. For example, a computer system can comprise one or more central processing units (CPU) and/or graphics processing units (GPU), input means, output means and data storage, which can be embodied as one or more connected computing devices. Preferably the computer system has a display or comprises a computing device that has a display to provide a visual output display. The data storage can comprise RAM, disk drives, solid-state disks or other computer readable media. The computer system can comprise a plurality of computing devices connected by a network and able to communicate with each other over that network. It is explicitly envisaged that computer system can consist of or comprise a cloud computer. The motion activity monitoring systems described herein can comprise wearable sensors comprising actigraph units, in particular wrist-worn wearable sensors comprising accelerometers such as smartwatches, and/or sensors integrated into objects that can be placed on the subject such as for example smartphones, tablets, laptops, and/or directly within the body, such as for example subcutaneous chips, and/or ballistocardiogram sensors such as for example electronic sleep mattresses.

As used herein “data” and “signals” are used interchangeably unless otherwise specified.

The methods described herein are computer implemented unless context indicates otherwise. Indeed, the features of the data associated with sleep motion activity patterns are such that the methods described herein are far beyond the capability of the human brain and can not be performed as a mental act. The methods described herein can be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media, magnetic tape; optical storage media such as optical discs or CD-ROMs; electrical storage media such as memory, including RAM, ROM and flash memory; hybrids and combinations of the above such as magnetic/optical storage media.

The invention relates to processing and/or statistical analysis of digital signals, for example to characterize motion activity patterns during sleep. As used herein “sleep motion activity” refers to motion activity during sleep. As used herein “sleep motion activity patterns” refer to patterns of motion activity during sleep. As used herein “patterns” refer to particular ways in which motion activity during sleep occurs and can comprise regular and/or irregular patterns. For example but by no way of limitation, patterns can comprise regular or irregular motion (relative) intensities, regular or irregular number of body movements, regular or irregular motion occurrences in time, motion intensities variability, variability of body movements. As used herein “activity levels” refer to levels of intensity of motion activity. They can be unitless numbers. Their absolute values can depend on the specific motion activity monitoring system used. Activity levels can be computed for a predetermined epoch. An epoch is a time slot of predetermined duration. As used herein “activity level profiles” refer to histograms of activity levels, in particular histograms of dB-converted activity levels binned in dB bins. The number of dB bins of an activity level profile can be chosen independently of the number of epochs for which original, i.e. non dB-converted, activity levels are computed.

As used herein “similarity” refers to the result of one or several combined similarity measurements between objects, in particular between activity level profiles or between activity levels in predetermined bins or between activity levels in predetermined epochs. Similarity measurements refer to statistical methods or statistical metrics that quantify the similarity between two mathematical objects. Cosine similarity is a commonly used similarity measurement for real-valued vectors. Intersection over Unit (IoU) is a known metric for measuring overlap between objects. Statistical tests can also be used as similarity measurements. For example, a p-value in a p-value test is the probability of occurrence of an event under the assumption of a null hypothesis. With a null hypothesis formulated as the hypothesis that two objects are equal, the p-value can be used as a probability of similarity between said objects.

### Systems

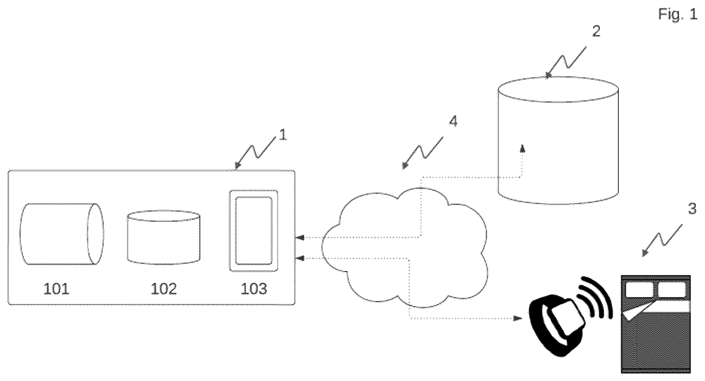

FIG. 1 illustrates an embodiment of a system that can be used to implement one or more aspects described herein. With reference to FIG. 1, the system comprises a computing device 1, which comprises a processor 101 and a computer readable memory 102. In the embodiment shown, the computing device 1 also comprises a user interface 103, which is illustrated as a screen but can include any other means of conveying information to a user such as e.g. through audible or visual signals. The computing device 1 is communicably connected, such as e.g. through a network, to one or more motion activity monitoring systems, such as actigraph units and/or ballistocardiogram sensors, and/or to one or more databases 2 storing signals from the one or more motion activity monitoring systems. The one or more databases 2 can further store one or more of: control data, parameters (such as e.g. thresholds derived from control data, parameters used for normalization, etc.), clinical and/or patient related information, etc. The computing device can be a smartphone, tablet, personal computer or other computing device. The computing device can be configured to implement a method of processing signals, as described herein. In alternative embodiments, the computing device 1 is configured to communicate with a remote computing device (not shown), which is itself configured to implement a method of processing signals from one or more motion activity monitoring systems as described herein. In such cases, the remote computing device can also be configured to send the result of the method of processing signals from one or more motion activity monitoring systems. Communication between the computing device 1 and the remote computing device can be through a wired or wireless connection, and can occur over a local or public network 4 such as e.g. over the public internet. The motion activity monitoring systems 3 can be in wired connection with the computing device 1, or can be able to communicate through a wireless connection, such as e.g. through WiFi and/or over the public internet, as illustrated. The connection between the computing device 1 and the motion activity monitoring systems 3 can be direct or indirect (such as e.g. through a remote computer). The motion activity monitoring systems 3 are configured to record signals related to motion activity of a subject. In some embodiments, the recorded signals can have been subject to one or more preprocessing steps (eg cropping, resizing, normalizing, etc) prior to performing the methods described herein.

### Methods

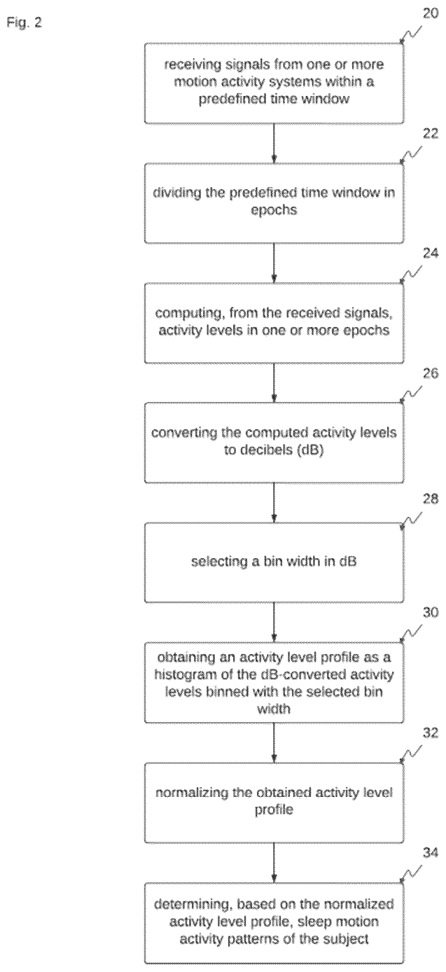

FIG. 2 is a flow diagram showing, in schematic form, a method of processing signals from one or more motion activity monitoring systems to determine sleep motion activity patterns of a subject, according to the invention. With reference to FIG. 2, at step 20 signals from one or more motion activity systems within a predefined time window are received. This comprises receiving signals actigraph units and/or ballistocardiogram sensors. The use of actigraph units can be advantageous since they are typically wrist-worn and allow to measure continuously body accelerations. The use of ballistocardiogram sensors such as for example electronic sleep mattresses can be advantageous since they are the least invasive and allow to detect small-intensity movements such as blood flow, heartbeat, respiratory movements. Signals can be received directly from the motion activity systems. Signals can have been previously recorded and received, for example from a memory or other computing devices. Signals can be collected at a predetermined sampling frequency, for example 200 Hz. Signals can be resampled at a predetermined frequency. Signals from different types of motion activity systems can be calibrated across devices. At step 22, the predefined time window is divided in epochs. For example, a “night” time window (i.e. 8 hours of sleep) can be divided in epochs with time duration of 4 seconds 425 each. At step 24, activity levels are computed in one or more epochs for the received signals. For example, within all or some of the 4-seconds epochs, a summarized metric of the signals can be calculated. A summarized metric can be for example a mean, a median, a trimmed version thereof, maximum, minimum, percentile. At step 26, the computed activity levels are converted to decibels (dB). For example, activity level x can be converted in dB using the transform 10*log₁₀ (αx+β), in particular wherein α=β=1. At step 28, a bin width in dB is selected. For example, a bin width of 1 dB can be selected. Alternatively, a bin width of 2 dB can be selected. Alternatively, a bin width of 5 dB can be selected. At step 30, an activity level profile is obtained as a histogram of the dB-converted activity levels binned with the selected bin width. At step 32, the obtained activity level profile is normalized. For example, the activity level profile can be normalized by computing the normalized activity level profile according to the equation

$${h}^{′} = {( {h}_{1} , … , {h}_{n} )}^{T} / {\sum }_{i = 1}^{n} {h}_{i} ,$$

where hᵢ is the value of i-th bin of the activity intensity histogram prior to normalization. At step 34, sleep motion activity patterns of the subject are determined based on the normalized activity level profile. For example, from the normalized activity level profiles the probabilities of occurrences of body movements with a given intensity can be calculated. Higher values in certain dB bins point to higher likelihood of occurrence of movements of the respective activity level.

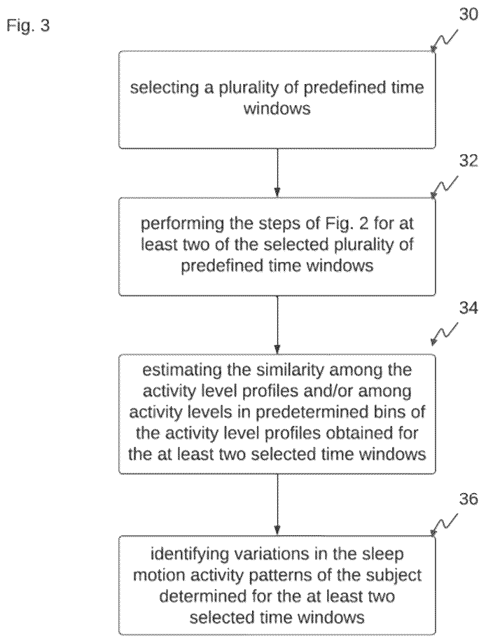

FIG. 3 is a flow diagram showing, in schematic form, a method of processing signals from one or more motion activity monitoring systems to determine sleep motion activity patterns of a subject for at least two selected time windows, according to the invention. With reference to FIG. 3, at step a plurality of predefined time windows is selected. For example, a plurality of “nights” time windows is selected over a full year. For example, a plurality of “nights” time windows is selected over multiple years. For example, a plurality of sequential “nights” time windows is selected. At step 32, for at least two of the selected plurality of predefined time windows the steps of FIG. 2 can be performed. At step 34, the similarity among the normalized activity level profiles and/or among activity levels in predetermined bins of the normalized activity level profiles obtained for the at least two selected time windows is estimated. In an embodiment, this step can comprise estimating the similarity among the normalized activity level profiles obtained for more than two time windows. In such embodiment, this step can comprise computing a summarized normalized activity level profile as a summarized metric of the normalized activity level profiles over time. For example, a weekly-mean distribution can be computed as the mean of the normalized activity level profiles of some or all nights in a time window of a week. For example, a monthly-mean distribution can be computed as the mean of the normalized activity level profiles of some or all nights in a time window of a month. At this step, the similarity can be estimated by performing one or more similarity measurements comprising cosine similarity measurement, Intersection over Unit measurement, statistical tests, in particular a p-value test, or a combination thereof. At step 36, variations in the sleep motion activity patterns of the subject determined for the at least two selected time windows are identified. For example, this can comprise measuring the variation of (relative) intensity and/or of number of body movements and/or of time occurrence of body movements.

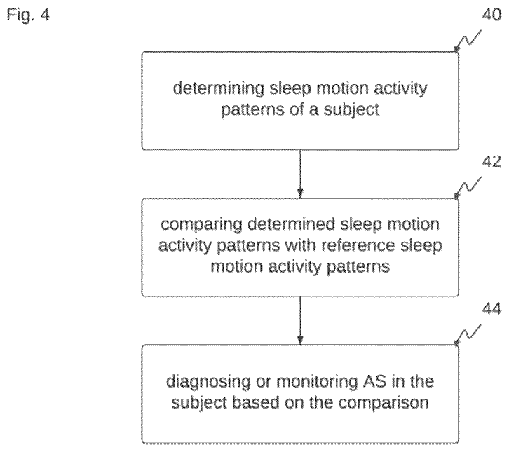

FIG. 4 is a flow diagram showing, in schematic form, a method of diagnosing or monitoring Angelman Syndrome in a subject, according to the invention. With reference to FIG. 4, at step 40 sleep motion activity patterns of a subject are determined according to the invention. At step 42, the determined sleep motion activity patterns are compared with reference sleep motion activity patterns. At step 44, Angelman Syndrome is diagnosed or monitored based on the comparison among sleep motion activity patterns.

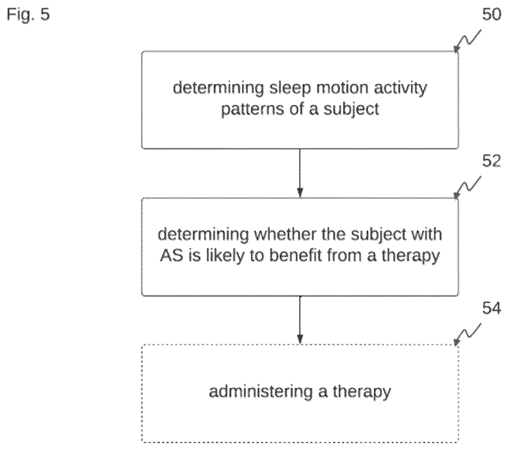

FIG. 5 is a flow diagram showing, in schematic form, a method of determining whether a subject with Angelman Syndrome is likely to benefit from a therapy, according to the invention. With reference to FIG. 5, at step 50 sleep motion activity patterns of a subject are determined according to the invention. At step 52, it is determined whether a subject with Angelman Syndrome is likely to benefit from a therapy. At optional step 54, the therapy is administered.

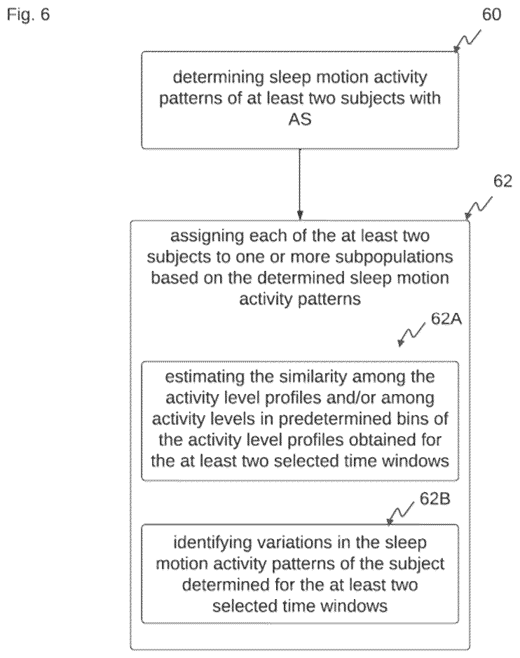

FIG. 6 is a flow diagram showing, in schematic form, a method of identifying one or more subpopulations of subjects with Angelman Syndrome, according to the invention. With reference to FIG. 6, at step 60 sleep motion activity patterns of at least two subjects with Angelman Syndrome are determined according to the invention. At step 62, each of the at least two subjects are assigned to the one or more subpopulations based on the determined sleep motion activity patterns. This step can comprise step 62A and/or step 62B. At step 62A, the similarity among the activity level profiles and/or among activity levels in predetermined bins of the activity level profiles obtained for the at least two selected time windows is estimated. At step 62B, variations in the sleep motion activity patterns of the subjects determined for the at least two selected time windows can be identified.

### EXAMPLES

The examples below illustrate applications of the methods of the present invention, in particular in the context of determining sleep motion activity patterns of subjects by processing signals from an electromechanical sleep mattress with the goal of diagnosing or monitoring sleep disturbances occurring in subjects with Angelman Syndrome. The electromechanical sleep mattress used was sensitive to high-intensity body movements, such as the movements of the arms, the legs, the head, movements due to seizures, repetitive movements such as repetitive leg movements, tosses and turns, as well as to low-intensity body movements, such as heartbeat, blood flow, respiratory movements, tiny vibrations.

Example 1 shows normalized activity level profiles of an 18-years old subject obtained by analyzing signals recorded with an electromechanical sleep mattress within several nights. Example 2 shows normalized activity level profiles of a 2-years old subject obtained by analyzing signals recorded with an electromechanical sleep mattress within several nights. Example 3 shows an example of patient stratification obtained by identifying subpopulations of subjects based on their sleep motion activity patterns.

### Example 1—Stable Sleep Motion Activity Patterns

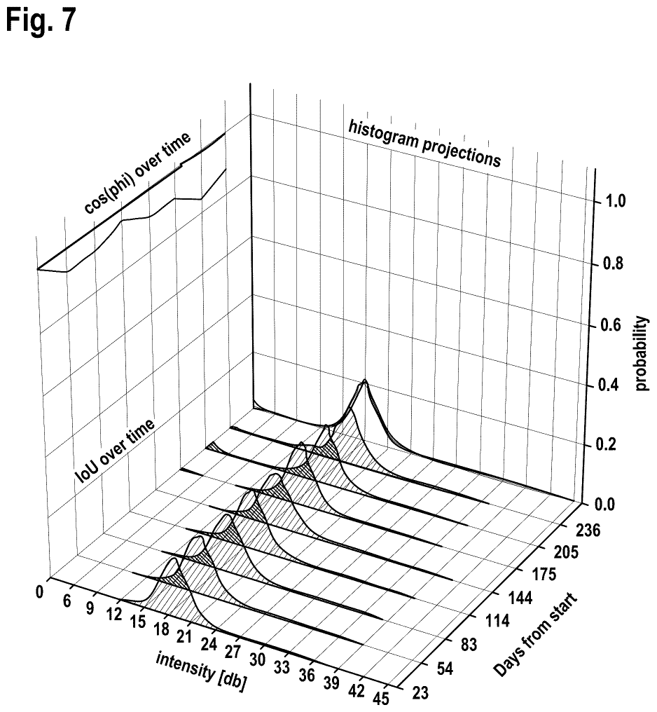

In this example, signals from an electromechanical sleep mattress were recorded for an 18-years old subject diagnosed with Angelman Syndrome. Signals were recorded for all nights during a time window of 8 months. Said signals were processed according to the invention. The resulting normalized activity level profiles obtained for all nights over a month were averaged to obtain monthly-means distributions. Monthly-means distributions are shown in FIG. 7. The sleep motion activity patterns of this subject reveal a predominance of likelihood of activity in the intensity range 15-20 dB. The similarity among normalized activity level profiles over time is measured via a cosine similarity and shown in the Figure. Stable sleep motion activity patterns, i.e. patterns that do not change over time as shown for this subject, can indicate that the disease status is stable.

### Example 2-Unstable Sleep Motion Activity Patterns

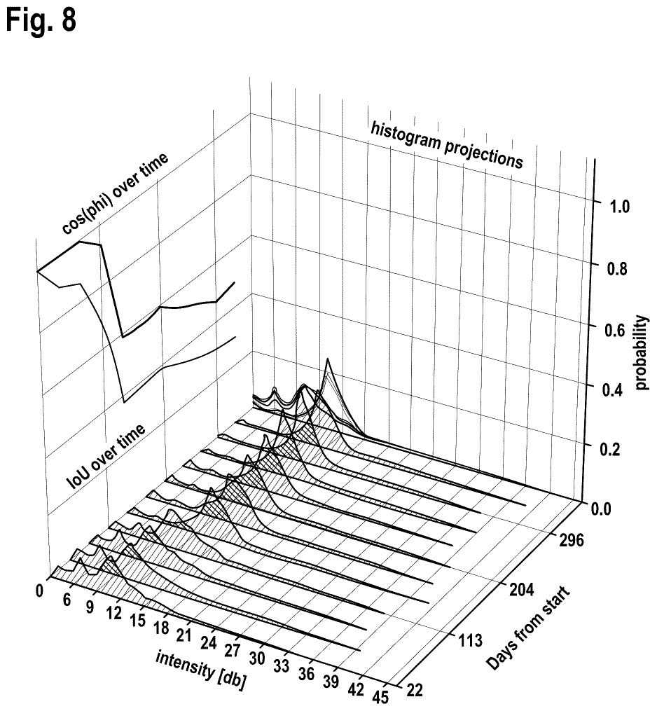

In this example, signals from an electromechanical sleep mattress were recorded for a 2-years old subject diagnosed with Angelman Syndrome. Signals were recorded for all nights during a time window of 11 months for the 2 years old subject. Said signals were processed according to the invention. The resulting normalized activity level profiles obtained for all nights over a month were averaged to obtain monthly-means distributions. Monthly-means distributions are shown in FIG. 8. The sleep motion activity patterns of this subject reveal a predominance of likelihood of activity in the intensity range 7-12 dB in the first 5 months and in the intensity range 12-15 dB in the last 6 months. The similarity among normalized activity level profiles over time is measured via a cosine similarity and shown in the Figure. Unstable sleep motion activity patterns, i.e. patterns that change over time as shown for this subject, can indicate that the disease is progressing, as in particular for Angelman Syndrome a higher-intensity sleep motion activity correlates with a worsening of the disease. However, time-abrupt changes in the peak of the monthly-means distributions can also indicate an effect of a medication, changes in the sleep environment, e.g. seasonal effect, etc.

### Example 3—Common and Distinguishing Features of Sleep Motion Activity Patterns Among Patient Cohorts

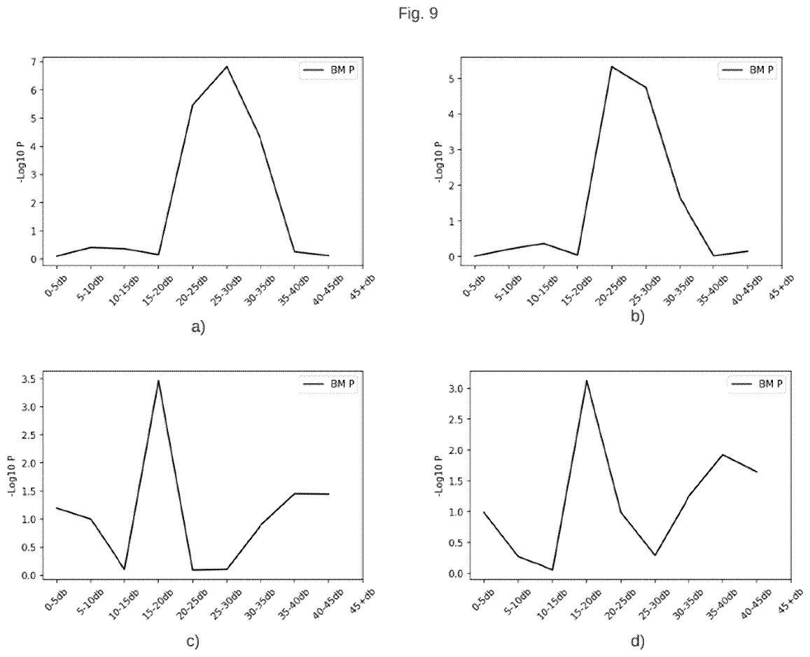

In this example, signals from an electromechanical sleep mattress were recorded for each of several subjects in the age range 1-12 years old, some of which were healthy subjects and some of which were diagnosed with Angelman Syndrome. Said signals were processed according to the invention. A non-parametric Brunner-Munzel (BM) statistical test was performed to estimate the similarity among normalized activity level profiles for all subjects. FIG. 9 shows p-values for 535 BM tests over different intensity levels. The y axis shows −Log₁₀ P, where P is the probability of similarity. A value of 3 on the y axis corresponds to a quite significant probability of similarity of 10⁻³. A value of 7 on the y axis corresponds to an extremely significant probability of similarity of 10⁻⁷. In other words, the higher the value on the y axis, the more dissimilar the distributions are. Results are shown for: 1-12 years old AS patients vs 1-12 years old healthy subjects (FIG. 9a), 1-5 years old AS patients vs 1-5 years old healthy subjects (FIG. 9b), 1-5 years old AS patients vs 6-12 years old AS patients (FIG. 9c), 1-5 years old healthy subjects vs 6-12 years old healthy subjects (FIG. 9d). Results show that differences in the intensity range of 15-20 dB can be associated with different age ranges (1-5 vs 6-12 years old), while differences in the intensity range of 25-30 dB can be associated with the presence or likelihood of being diagnosed with Angelman Syndrome. Movements in the intensity range of 25 dB and above can correspond to tosses and turns.

### Embodiments

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

The methods of any embodiments described herein may be provided as computer programs or as computer program products or computer readable media carrying a computer program which is arranged, when run on a computer, to perform the method(s) described above.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. In an embodiment, a computer-implemented method of processing signals from one or more motion activity monitoring systems to determine sleep motion activity patterns of a subject is disclosed, the method comprising the steps of:
a. receiving signals from the one or more motion activity monitoring systems within a predefined time window, wherein the one or more motion activity monitoring systems comprise actigraph units and/or ballistocardiogram sensors;
b. dividing the predefined time window in epochs;
c. computing, from the received signals, activity levels in one or more epochs;
d. converting the computed activity levels in decibels (dB);
e. selecting a bin width in dB;
f. obtaining an activity level profile as a histogram of the decibels-converted activity levels binned with the selected bin width;
g. normalizing the obtained activity level profile;
h. determining, based on the normalized activity level profile, the sleep motion activity patterns of the subject.

2. In an embodiment, a computer-implemented method of processing signals from one or more motion activity monitoring systems to determine sleep motion activity patterns of a subject is disclosed, the method comprising the steps of:
a. receiving signals from the one or more motion activity monitoring systems within a predefined time window, wherein the one or more motion activity monitoring systems comprise actigraph units or ballistocardiogram sensors;
b. dividing the predefined time window in epochs;
c. computing, from the received signals, activity levels in one or more epochs;
d. converting the computed activity levels in decibels (dB);
e. selecting a bin width in dB;
f. obtaining an activity level profile as a histogram of the decibels-converted activity levels binned with the selected bin width;
g. normalizing the obtained activity level profile;
h. determining, based on the normalized activity level profile, the sleep motion activity patterns of the subject.

3. In an embodiment, a computer-implemented method of processing signals from one or more motion activity monitoring systems to determine sleep motion activity patterns of a subject is disclosed, the method comprising the steps of:
a. receiving signals from the one or more motion activity monitoring systems within a predefined time window, wherein the one or more motion activity monitoring systems comprise actigraph units and ballistocardiogram sensors;
b. dividing the predefined time window in epochs;
c. computing, from the received signals, activity levels in one or more epochs;
d. converting the computed activity levels in decibels (dB);
e. selecting a bin width in dB;
f. obtaining an activity level profile as a histogram of the decibels-converted activity levels binned with the selected bin width;
g. normalizing the obtained activity level profile;
h. determining, based on the normalized activity level profile, the sleep motion activity patterns of the subject.

4. In an embodiment, a computer-implemented method of processing signals from one or more motion activity monitoring systems to determine sleep motion activity patterns of a subject is disclosed, the method comprising the steps of:
a. receiving signals from the one or more motion activity monitoring systems within a predefined time window, wherein the one or more motion activity monitoring systems comprise actigraph units and/or ballistocardiogram sensors;
b. dividing the predefined time window in epochs;
c. computing, from the received signals, activity levels in one or more epochs;
d. converting the computed activity levels in decibels (dB);
e. selecting a bin width in dB;
f. obtaining an activity level profile as a histogram of the decibels-converted activity levels binned with the selected bin width;
g. determining, based on the activity level profile, the sleep motion activity patterns of the subject.

5. In an embodiment, a computer-implemented method of processing signals from one or more motion activity monitoring systems to determine sleep motion activity patterns of a subject is disclosed, the method comprising the steps of:
a. receiving signals from the one or more motion activity monitoring systems within a predefined time window, wherein the one or more motion activity monitoring systems comprise actigraph units or ballistocardiogram sensors;
b. dividing the predefined time window in epochs;
c. computing, from the received signals, activity levels in one or more epochs;
d. converting the computed activity levels in decibels (dB);
e. selecting a bin width in dB;
f. obtaining an activity level profile as a histogram of the decibels-converted activity levels binned with the selected bin width;
g. determining, based on the activity level profile, the sleep motion activity patterns of the subject.

6. In an embodiment, a computer-implemented method of processing signals from one or more motion activity monitoring systems to determine sleep motion activity patterns of a subject is disclosed, the method comprising the steps of:
a. receiving signals from the one or more motion activity monitoring systems within a predefined time window, wherein the one or more motion activity monitoring systems comprise actigraph units and ballistocardiogram sensors;
b. dividing the predefined time window in epochs;
c. computing, from the received signals, activity levels in one or more epochs;
d. converting the computed activity levels in decibels (dB);
e. selecting a bin width in dB;
f. obtaining an activity level profile as a histogram of the decibels-converted activity levels binned with the selected bin width;
g. determining, based on the activity level profile, the sleep motion activity patterns of the subject.

7. In an embodiment, the method of any preceding embodiments is disclosed, wherein the epochs comprise time slots of duration between 1 second and 60 seconds, in particular 4 seconds.

8. In an embodiment, the method of any of embodiments 1-6 is disclosed, wherein the epochs comprise time slots of duration of 1 second.

9. In an embodiment, the method of any of embodiments 1-6 is disclosed, wherein the epochs comprise time slots of duration of 60 seconds.

10. In an embodiment, the method of any of embodiments 1-6 is disclosed, wherein the epochs comprise time slots of duration of 4 seconds.

11. In an embodiment, the method of any preceding embodiments is disclosed, wherein converting the computed activity levels in dB comprises converting activity level x using the transform 10*log₁₀ (αx+β), in particular wherein α=β=1.

12. In an embodiment, the method of any of embodiments 1-10 is disclosed, wherein converting the computing activity levels in dB comprises converting activity level x using the transform 10*log₁₀ (αx+β).

13. In an embodiment, the method of any preceding embodiments is disclosed, wherein selecting a bin width in dB comprises selecting a bin width of 1 dB, of 2 dB, of 5 dB.

14. In an embodiment, the method of any of embodiments 1-12 is disclosed, wherein selecting a bin width in dB comprises selecting a bin width of 1 dB.

15. In an embodiment, the method of any of embodiments 1-12 is disclosed, wherein selecting a bin width in dB comprises selecting a bin width of 2 dB.

16. In an embodiment, the method of any of embodiments 1-12 is disclosed, wherein selecting a bin width in dB comprises selecting a bin width of 5 dB.

17. In an embodiment, the method of any preceding embodiments is disclosed, wherein normalizing the activity level profile comprises computing the normalized activity level profile according to the equation

$${h}^{′} = {( {h}_{1} , … , {h}_{n} )}^{T} / {\sum }_{i = 1}^{n} {h}_{i} ,$$

where hᵢ is the value of the activity level in the i-th bin.

18. In an embodiment, the method of any preceding embodiments is disclosed, wherein determining, based on the normalized activity level profile, the sleep motion activity patterns of the subject comprises measuring the relative intensity and/or the number of body movements.

19. In an embodiment, the method of any of embodiments 1-17 is disclosed, wherein determining, based on the normalized activity level profile, the sleep motion activity patterns of the subject comprises measuring the relative intensity or the number of body movements.

20. In an embodiment, the method of any of embodiments 1-17 is disclosed, wherein determining, based on the normalized activity level profile, the sleep motion activity patterns of the subject comprises measuring the relative intensity and the number of body movements.

21. In an embodiment, the method of any of embodiments 1-17 is disclosed, wherein determining, based on the activity level profile, the sleep motion activity patterns of the subject comprises measuring the intensity and/or the number of body movements.

22. In an embodiment, the method of any of embodiments 1-17 is disclosed, wherein determining, based on the activity level profile, the sleep motion activity patterns of the subject comprises measuring the intensity or the number of body movements.

23. In an embodiment, the method of any of embodiments 1-17 is disclosed, wherein determining, based on the activity level profile, the sleep motion activity patterns of the subject comprises measuring the intensity and the number of body movements.

24. In an embodiment, the method of any preceding embodiments is disclosed, further comprising selecting a plurality of predefined time windows and, for at least two of the selected time windows, performing the steps a. to h.

25. In an embodiment, the method of any preceding embodiments is disclosed, further comprising selecting a plurality of predefined time windows and, for at least two of the selected time windows, performing some of the steps a. to g.

26. In an embodiment, the method of any preceding embodiments is disclosed, further comprising selecting a plurality of predefined time windows and, for two of the selected time windows, performing the steps a. to h.

27. In an embodiment, the method of any preceding embodiments is disclosed, further comprising selecting a plurality of predefined time windows and, for two of the selected time windows, performing some of the steps a. to g.

28. In an embodiment, the method of any of embodiments 24-27 is disclosed, further comprising the steps of: estimating the similarity among the normalized activity level profiles and/or among activity levels in predetermined bins of the normalized activity level profiles obtained for the at least two selected time windows; and/or identifying variations in the sleep motion activity patterns of the subject determined for the at least two selected time windows.

29. In an embodiment, the method of any of embodiments 24-27 is disclosed, further comprising the steps of: estimating the similarity among the normalized activity level profiles or among activity levels in predetermined bins of the normalized activity level profiles obtained for the at least two selected time windows; and/or identifying variations in the sleep motion activity patterns of the subject determined for the at least two selected time windows.

30. In an embodiment, the method of any of embodiments 24-27 is disclosed, further comprising the steps of: estimating the similarity among the normalized activity level profiles and among activity levels in predetermined bins of the normalized activity level profiles obtained for the at least two selected time windows; and/or identifying variations in the sleep motion activity patterns of the subject determined for the at least two selected time windows.

31. In an embodiment, the method of any of embodiments 24-27 is disclosed, further comprising the steps of: estimating the similarity among the normalized activity level profiles and/or among activity levels in predetermined bins of the normalized activity level profiles obtained for the at least two selected time windows; or identifying variations in the sleep motion activity patterns of the subject determined for the at least two selected time windows.

32. In an embodiment, the method of any of embodiments 24-27 is disclosed, further comprising the steps of: estimating the similarity among the normalized activity level profiles and/or among activity levels in predetermined bins of the normalized activity level profiles obtained for the at least two selected time windows; and identifying variations in the sleep motion activity patterns of the subject determined for the at least two selected time windows.

33. In an embodiment, the method of any of embodiments 24-27 is disclosed, further comprising the steps of: estimating the similarity among the normalized activity level profiles and among activity levels in predetermined bins of the normalized activity level profiles obtained for the at least two selected time windows; or identifying variations in the sleep motion activity patterns of the subject determined for the at least two selected time windows.

34. In an embodiment, the method of any of embodiments 24-27 is disclosed, further comprising the steps of: estimating the similarity among the normalized activity level profiles and among activity levels in predetermined bins of the normalized activity level profiles obtained for the at least two selected time windows; and identifying variations in the sleep motion activity patterns of the subject determined for the at least two selected time windows.

35. In an embodiment, the method of any of embodiments 24-27 is disclosed, further comprising the steps of: estimating the similarity among the normalized activity level profiles or among activity levels in predetermined bins of the normalized activity level profiles obtained for the at least two selected time windows; or identifying variations in the sleep motion activity patterns of the subject determined for the at least two selected time windows.

36. In an embodiment, the method of any of embodiments 24-27 is disclosed, further comprising the steps of: estimating the similarity among the normalized activity level profiles or among activity levels in predetermined bins of the normalized activity level profiles obtained for the at least two selected time windows; and identifying variations in the sleep motion activity patterns of the subject determined for the at least two selected time windows.

37. In an embodiment, the method of any of embodiments 24-27 is disclosed, further comprising the steps of: estimating the similarity among the activity level profiles and/or among activity levels in predetermined bins of the activity level profiles obtained for the at least two selected time windows; and/or identifying variations in the sleep motion activity patterns of the subject determined for the at least two selected time windows.

38. In an embodiment, the method of any of embodiments 24-27 is disclosed, further comprising the steps of: estimating the similarity among the activity level profiles or among activity levels in predetermined bins of the activity level profiles obtained for the at least two selected time windows; and/or identifying variations in the sleep motion activity patterns of the subject determined for the at least two selected time windows.

39. In an embodiment, the method of any of embodiments 24-27 is disclosed, further comprising the steps of: estimating the similarity among the activity level profiles and among activity levels in predetermined bins of the activity level profiles obtained for the at least two selected time windows; and/or identifying variations in the sleep motion activity patterns of the subject determined for the at least two selected time windows.

40. In an embodiment, the method of any of embodiments 24-27 is disclosed, further comprising the steps of: estimating the similarity among the activity level profiles and/or among activity levels in predetermined bins of the activity level profiles obtained for the at least two selected time windows; or identifying variations in the sleep motion activity patterns of the subject determined for the at least two selected time windows.

41. In an embodiment, the method of any of embodiments 24-27 is disclosed, further comprising the steps of: estimating the similarity among the activity level profiles and/or among activity levels in predetermined bins of the activity level profiles obtained for the at least two selected time windows; and identifying variations in the sleep motion activity patterns of the subject determined for the at least two selected time windows.

42. In an embodiment, the method of any of embodiments 24-27 is disclosed, further comprising the steps of: estimating the similarity among the activity level profiles and among activity levels in predetermined bins of the activity level profiles obtained for the at least two selected time windows; or identifying variations in the sleep motion activity patterns of the subject determined for the at least two selected time windows.

43. In an embodiment, the method of any of embodiments 24-27 is disclosed, further comprising the steps of: estimating the similarity among the activity level profiles and among activity levels in predetermined bins of the activity level profiles obtained for the at least two selected time windows; and identifying variations in the sleep motion activity patterns of the subject determined for the at least two selected time windows.

44. In an embodiment, the method of any of embodiments 24-27 is disclosed, further comprising the steps of: estimating the similarity among the activity level profiles or among activity levels in predetermined bins of the activity level profiles obtained for the at least two selected time windows; or identifying variations in the sleep motion activity patterns of the subject determined for the at least two selected time windows.

45. In an embodiment, the method of any of embodiments 24-27 is disclosed, further comprising the steps of: estimating the similarity among the activity level profiles or among activity levels in predetermined bins of the activity level profiles obtained for the at least two selected time windows; and identifying variations in the sleep motion activity patterns of the subject determined for the at least two selected time windows.

46. In an embodiment, the method of any of embodiments 28-45 is disclosed, wherein estimating the similarity comprises performing one or more similarity measurements comprising cosine similarity measurement, Intersection over Unit measurement, statistical tests, in particular p-value tests, or a combination thereof.

47. In an embodiment, the method of any of embodiments 28-46 is disclosed, wherein identifying variations in the sleep motion activity patterns of the subject comprises measuring the variation of relative intensity and/or of number of body movements and/or of time occurrence of body movements.

48. In an embodiment, the method of any of embodiments 28-46 is disclosed, wherein identifying variations in the sleep motion activity patterns of the subject comprises measuring the variation of relative intensity or of number of body movements or of time occurrence of body movements.

49. In an embodiment, the method of any of embodiments 28-46 is disclosed, wherein identifying variations in the sleep motion activity patterns of the subject comprises measuring the variation of relative intensity and of number of body movements and of time occurrence of body movements.

50. In an embodiment, the method of any of embodiments 28-46 is disclosed, wherein identifying variations in the sleep motion activity patterns of the subject comprises measuring the variation of intensity and/or of number of body movements and/or of time occurrence of body movements.

51. In an embodiment, the method of any of embodiments 28-46 is disclosed, wherein identifying variations in the sleep motion activity patterns of the subject comprises measuring the variation of intensity or of number of body movements or of time occurrence of body movements.

52. In an embodiment, the method of any of embodiments 28-46 is disclosed, wherein identifying variations in the sleep motion activity patterns of the subject comprises measuring the variation of intensity and of number of body movements and of time occurrence of body movements.

53. In an embodiment, a method of diagnosing or monitoring a neurological dysfunction associated with sleep motion activity in a subject is disclosed, the method comprising determining the sleep motion activity patterns of a subject using the method of any of the preceding embodiments, optionally wherein the neurological dysfunction is Angelman Syndrome (AS).

54. In an embodiment, a method of diagnosing and monitoring a neurological dysfunction associated with sleep motion activity in a subject is disclosed, the method comprising determining the sleep motion activity patterns of a subject using the method of any of the preceding embodiments, optionally wherein the neurological dysfunction is Angelman Syndrome (AS).

55. In an embodiment, a method of diagnosing or monitoring a neurological dysfunction associated with sleep motion activity in a subject is disclosed, the method comprising determining the sleep motion activity patterns of a subject using the method of any of the preceding embodiments.

56. In an embodiment, a method of diagnosing and monitoring a neurological dysfunction associated with sleep motion activity in a subject is disclosed, the method comprising determining the sleep motion activity patterns of a subject using the method of any of the preceding embodiments.

57. In an embodiment, a method of determining whether a subject with a neurological dysfunction associated with sleep motion activity is likely to benefit from a therapy is disclosed, the method comprising determining the sleep motion activity patterns of a subject using the method of any of the preceding embodiments, optionally wherein the neurological dysfunction is Angelman Syndrome (AS).

58. In an embodiment, a method of determining whether a subject with a neurological dysfunction associated with sleep motion activity is likely to benefit from a therapy is disclosed, the method comprising determining the sleep motion activity patterns of a subject using the method of any of the preceding embodiments.

59. In an embodiment, a method of identifying one or more subpopulations of subjects with a neurological dysfunction associated with sleep motion activity is disclosed, the method comprising determining the sleep motion activity patterns of at least two of the subjects using the method of any of the preceding embodiments, and assigning each of the at least two subjects to the one or more subpopulations based on the determined sleep motion activity patterns, optionally wherein the neurological dysfunction is Angelman Syndrome (AS).

60. In an embodiment, a method of identifying one or more subpopulations of subjects with a neurological dysfunction associated with sleep motion activity is disclosed, the method comprising determining the sleep motion activity patterns of at least two of the subjects using the method of any of the preceding embodiments, and assigning some of the at least two subjects to the one or more subpopulations based on the determined sleep motion activity patterns, optionally wherein the neurological dysfunction is Angelman Syndrome (AS).

61. In an embodiment, a method of identifying one or more subpopulations of subjects with a neurological dysfunction associated with sleep motion activity is disclosed, the method comprising determining the sleep motion activity patterns of at least two of the subjects using the method of any of the preceding embodiments.

62. In an embodiment, the method of embodiment 59 disclosed, wherein assigning each of the at least two subjects to the one or more subpopulations based on the determined sleep motion activity patterns comprises: estimating the similarity among the normalized activity level profiles and/or among activity levels in predetermined bins of the normalized activity level profiles obtained for the at least two subjects; and/or identifying variations in the sleep motion activity patterns determined for the at least two subjects.

63. In an embodiment, the method of embodiment 59 disclosed, wherein assigning each of the at least two subjects to the one or more subpopulations based on the determined sleep motion activity patterns comprises: estimating the similarity among the normalized activity level profiles and/or among activity levels in predetermined bins of the normalized activity level profiles obtained for the at least two subjects; or identifying variations in the sleep motion activity patterns determined for the at least two subjects.

64. In an embodiment, the method of embodiment 59 disclosed, wherein assigning each of the at least two subjects to the one or more subpopulations based on the determined sleep motion activity patterns comprises: estimating the similarity among the normalized activity level profiles and/or among activity levels in predetermined bins of the normalized activity level profiles obtained for the at least two subjects; and identifying variations in the sleep motion activity patterns determined for the at least two subjects.

65. In an embodiment, the method of embodiment 59 disclosed, wherein assigning each of the at least two subjects to the one or more subpopulations based on the determined sleep motion activity patterns comprises: estimating the similarity among the activity level profiles and/or among activity levels in predetermined bins of the activity level profiles obtained for the at least two subjects; and/or identifying variations in the sleep motion activity patterns determined for the at least two subjects.

66. In an embodiment, the method of embodiment 59 disclosed, wherein assigning each of the at least two subjects to the one or more subpopulations based on the determined sleep motion activity patterns comprises: estimating the similarity among the activity level profiles and/or among activity levels in predetermined bins of the activity level profiles obtained for the at least two subjects; or identifying variations in the sleep motion activity patterns determined for the at least two subjects.

67. In an embodiment, the method of embodiment 59 disclosed, wherein assigning each of the at least two subjects to the one or more subpopulations based on the determined sleep motion activity patterns comprises: estimating the similarity among the activity level profiles and/or among activity levels in predetermined bins of the activity level profiles obtained for the at least two subjects; and identifying variations in the sleep motion activity patterns determined for the at least two subjects.

68. In an embodiment, the method of embodiment 60 disclosed, wherein assigning some of the at least two subjects to the one or more subpopulations based on the determined sleep motion activity patterns comprises: estimating the similarity among the normalized activity level profiles and/or among activity levels in predetermined bins of the normalized activity level profiles obtained for the at least two subjects; and/or identifying variations in the sleep motion activity patterns determined for the at least two subjects.

69. In an embodiment, the method of embodiment 59 disclosed, wherein assigning some of the at least two subjects to the one or more subpopulations based on the determined sleep motion activity patterns comprises: estimating the similarity among the normalized activity level profiles and/or among activity levels in predetermined bins of the normalized activity level profiles obtained for the at least two subjects; or identifying variations in the sleep motion activity patterns determined for the at least two subjects.

70. In an embodiment, the method of embodiment 59 disclosed, wherein assigning some of the at least two subjects to the one or more subpopulations based on the determined sleep motion activity patterns comprises: estimating the similarity among the normalized activity level profiles and/or among activity levels in predetermined bins of the normalized activity level profiles obtained for the at least two subjects; and identifying variations in the sleep motion activity patterns determined for the at least two subjects.

71. In an embodiment, the method of embodiment 59 disclosed, wherein assigning some of the at least two subjects to the one or more subpopulations based on the determined sleep motion activity patterns comprises: estimating the similarity among the activity level profiles and/or among activity levels in predetermined bins of the activity level profiles obtained for the at least two subjects; and/or identifying variations in the sleep motion activity patterns determined for the at least two subjects.

72. In an embodiment, the method of embodiment 59 disclosed, wherein assigning some of the at least two subjects to the one or more subpopulations based on the determined sleep motion activity patterns comprises: estimating the similarity among the activity level profiles and/or among activity levels in predetermined bins of the activity level profiles obtained for the at least two subjects; or identifying variations in the sleep motion activity patterns determined for the at least two subjects.

73. In an embodiment, the method of embodiment 59 disclosed, wherein assigning some of the at least two subjects to the one or more subpopulations based on the determined sleep motion activity patterns comprises: estimating the similarity among the activity level profiles and/or among activity levels in predetermined bins of the activity level profiles obtained for the at least two subjects; and identifying variations in the sleep motion activity patterns determined for the at least two subjects.

74. In an embodiment, a system is disclosed, comprising:
a. a processor; and
b. a computer readable medium comprising instructions that, when executed by the processor, cause the processor to perform the steps of the method of any of the preceding embodiments;
c. optionally one or more motion activity monitoring systems.

75. In an embodiment, a system is disclosed, comprising:
a. a processor; and
b. a computer readable medium comprising instructions that, when executed by the processor, cause the processor to perform the steps of the method of any of the preceding embodiments.

76. In an embodiment, a computer readable [storage] medium/data carrier is disclosed, comprising instructions that, when executed by a computer, cause the computer to carry out the steps of the method of any of the preceding embodiments.

77. In an embodiment, a computer program [product] is disclosed comprising instructions which, when the program is executed by a computer, cause the computer to carry out the steps of the method of any of the preceding embodiments.

78. In an embodiment, the invention as hereinbefore described is disclosed.

### REFERENCES

All references cited herein are incorporated herein by reference in their entirety and for all purposes to the same extent as if each individual publication or patent or patent application was specifically and individually indicated to be incorporated by reference in its entirety.
Long et al (2017), Actigraphy-based sleep/wake detection for insomniacs, IEEE 14th International Conference on Wearable and Implantable Body Sensor Networks (BSN).
Granovsky et al (2018), Actigraphy-based Sleep/Wake Pattern Detection using Convolutional 1005 Neural Networks, arXiv: 1802:07945v1.

1. A computer-implemented method of processing signals from one or more motion activity monitoring systems to determine sleep motion activity patterns of a subject, the method comprising the steps of:
a. receiving signals from the one or more motion activity monitoring systems within a predefined time window (20), wherein the one or more motion activity monitoring systems comprise actigraph units and/or ballistocardiogram sensors;
b. dividing the predefined time window in epochs (22);
c. computing, from the received signals, activity levels in one or more epochs (24);
d. converting the computed activity levels to decibels (dB) (26);
e. selecting a bin width in dB (28);
f. obtaining an activity level profile as a histogram of the decibels-converted activity levels binned with the selected bin width (30);
g. normalizing the obtained activity level profile (32);
h. determining, based on the normalized activity level profile, the sleep motion activity patterns of the subject (34).

2. The method of claim 1, wherein the epochs comprise time slots of duration between 1 second and 60 seconds, in particular 4 seconds.

3. The method of claim 1, wherein converting the computed activity levels in dB comprises converting activity level x using the transform 10*log₁₀ (αx+β), in particular wherein α=β=1.

4. The method of claim 1, wherein selecting a bin width in dB comprises selecting a bin width of 1 dB, of 2 dB, of 5 dB.

5. The method of claim 1, wherein normalizing the activity level profile comprises computing the normalized activity level profile according to the equation
$${h}^{′} = {( {h}_{1} , … , {h}_{n} )}^{T} / {\sum }_{i = 1}^{n} {h}_{i} ,$$
where hᵢ is the value of the activity level in the i-th bin.

6. The method of claim 1, wherein determining, based on the normalized activity level profile, the sleep motion activity patterns of the subject comprises measuring the relative intensity and/or the number of body movements.

7. The method of claim 1, further comprising selecting a plurality of predefined time windows and, for at least two of the selected time windows, performing the steps a. to h.

8. The method of claim 7, further comprising the steps of: estimating the similarity among the normalized activity level profiles and/or among activity levels in predetermined bins of the normalized activity level profiles obtained for the at least two selected time windows; and/or identifying variations in the sleep motion activity patterns of the subject determined for the at least two selected time windows.

9. The method of claim 8, wherein estimating the similarity comprises performing one or more similarity measurements comprising cosine similarity measurement, Intersection over Unit measurement, statistical tests, in particular p-value tests, or a combination thereof.

10. The method of claim 8, wherein identifying variations in the sleep motion activity patterns of the subject comprises measuring the variation of relative intensity and/or of number of body movements and/or of time occurrence of body movements.

11. (canceled)

12. (canceled)

13. The method of claim 1, further comprising identifying one or more subpopulations of subjects with a neurological dysfunction associated with sleep motion activity, by determining the sleep motion activity patterns of at least two of the subjects, and assigning each of the at least two subjects to the one or more subpopulations based on the determined sleep motion activity patterns.

14. The method of claim 13, wherein assigning each of the at least two subjects to the one or more subpopulations based on the determined sleep motion activity patterns comprises: estimating the similarity among normalized activity level profiles and/or among activity levels in predetermined bins of the normalized activity level profiles obtained for the at least two subjects; and/or identifying variations in the sleep motion activity patterns determined for the at least two subjects.

15. A system comprising:
a. a processor; and
b. a computer readable medium comprising instructions that, when executed by the processor, cause the processor to perform the steps of the method of claim 1;
c. optionally one or more motion activity monitoring systems.
